(19) United States
(12) Patent Application Publication
(10) Pub. No.: US 2023/0420089 A1
(43) Pub. Date: Dec. 28, 2023

(54) SYNTHETICALLY GENERATED HEALTHCARE DOCUMENTS FOR CLASSIFIER TRAINING

(71) Applicant: Concord III, LLC, Seattle, WA (US)

(72) Inventors: Andre Sublett, Bainbridge Island, WA (US); Tim Osten, Poulsbo, WA (US); John Scott, Stephenville, TX (US)

(73) Assignee: Concord III, LLC, Seattle, WA (US)

(21) Appl. No.: 17/846,113

(22) Filed: Jun. 22, 2022

(51) Int. Cl. G16H 10/60; G16H 50/70; G06N 20/00

(52) U.S. Cl. CPC G16H 10/60; G16H 50/70; G06N 20/00

(57) ABSTRACT

A synthetic generation of healthcare documents for use in training a classifier is described herein. Initially, a multiplicity of electronic forms are received in memory of a host computing system and data extracted from a specific common field located in each of the forms. A statistical metric is then computed for the specific common field a value synthetically generated for the specific common field according to the computed statistical metric. Finally, the synthetically generated value is inserted into the specific common field of a training version of the electronic forms and the training version of the electronic forms persisted as part of a training data set for a classifier adapted to classify the multiplicity of electronic forms.

### BACKGROUND OF THE INVENTION

### Field of the Invention

The present invention relates to the technical field of document processing and more particularly to the training of a classifier adapted to batch classify healthcare documents.

### Description of the Related Art

The exchange of forms-based health care documents amongst health care providers, insurers, patients and the like remains trapped in a universe of heterogeneous and uncoordinated co-dependent computing systems, with different parties to the delivery of health care to a patient providing and receiving health care information according to different standard formats and utilizing different modes of document exchange, ranging from traditional fax to cutting edge wireless device to device transmission. Indeed, owing to the wide disparity in technical sophistication between different actors in the healthcare environment, the fax remains critical as the lingua franca technology of information exchange.

Healthcare information differs from traditional information in that there exists a strict regulatory climate for the security of personal healthcare information (PHI). However, in so far as the use of fax is prevalent in the exchange of healthcare information, using automated text processing methods requires first the conversion of the fax image to text, then the optical character recognition (OCR) of the converted text only then followed by the execution of program logic designed to identify PHI. High speed processing of batches of fax documents, though, does not lend itself well to the simple OCR, parsing and recognition of PHI—especially, when the structure of a received fax representative of a forms-based document is not known a priori.

Modern techniques in high-speed batch processing of fax images address the computationally expensive process of OCR, parsing and recognition through the utilization of machine learning classifiers trained in the characterization of a format of a forms-based document so that the fields of the document known to have an association with PHI can be rapidly located and the content redacted or replaced from fictitious data so as to ensure compliance with those healthcare privacy regulations affecting the processing of PHI. Of course, in order to train a machine learning classifier to properly classify the formatting of a forms-based document, actual forms-based documents must be annotated for ground truth during the training process. The very act, however, of training the machine learning classifier, then, can result in an unintentional disclosure of PHI present in the training set of documents.

To account for the risk of the inadvertent disclosure of PHI in a training set of healthcare documentation, oftentimes artificially generated documents are used in the course of training the classifier. However, care must be taken to include data in each document which reflects reality and absolutely avoids arbitrariness. For instance, a person seeking treatment for a disease prevalent amongst a particular gender should also include a name that is consistent with the gender, and a person living in a particular region should receive treatment from a facility proximate to that region, and a person seeking treatment for a particular condition should also have an age of the typical patient experiencing the particular condition. Hence, randomized data will act to produce an unrealistic document resulting in an improperly trained classifier.

### BRIEF SUMMARY OF THE INVENTION

Embodiments of the present invention address technical deficiencies of the art in respect to the generation of large sets of realistic artificial documents for the purpose of training a classifier. To that end, embodiments of the present invention provides for a novel and non-obvious method for the synthetic generation of healthcare documents for use in training a classifier. Embodiments of the present invention also provide for a novel and non-obvious computing device adapted to perform the foregoing method. Finally, embodiments of the present invention provide for a novel and non-obvious data processing system incorporating the foregoing device in order to perform the foregoing method.

In one embodiment of the invention, a method for the synthetic generation of healthcare documents for use in training a classifier includes receiving a multiplicity of electronic forms in memory of a host computing system and extracting data from a specific common field located in each of the forms. A statistical metric is then computed for the specific common field a value synthetically generated for the specific common field according to the computed statistical metric. Finally, the synthetically generated value is inserted into the specific common field of a training version of the electronic forms and the training version of the electronic forms persisted as part of a training data set for a classifier adapted to classify the multiplicity of electronic forms. In one aspect of the embodiment, the electronic forms conform to an annotated template including an identification of the specific common field. In another aspect of the embodiment, random noise is generated and the synthetically generated value modified with the random noise. In even yet another aspect of the embodiment, the computed statistical metric is a distribution of values for the specific common field.

In another embodiment of the invention, a data processing system is adapted for synthetically generating health care forms for use in training a health care form classifier. The system includes a host computing having one or more computers, each with memory and one or processing units including one or more processing cores. The system further includes a synthetic form generation module. The module includes computer program instructions enabled while executing in the memory of at least one of the processing units of the host computing platform to receive a multiplicity of electronic forms in memory of a host computing system and extract data from a specific common field located in each of the forms, compute a statistical metric for the specific common field. The program instructions additionally synthetically generate a value for the specific common field according to the computed statistical metric, insert the synthetically generated value into the specific common field of a training version of the electronic forms and persist the training version of the electronic forms as part of a training data set for a classifier adapted to classify the multiplicity of electronic forms.

In this way, the technical deficiencies of the creation of a training data set for a healthcare document classifier are overcome owing to incorporation into a synthetic healthcare training document of statistically relevant values for different fields of data within the document. Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a pictorial illustration reflecting different aspects of a process of synthetically generating healthcare documents for use in training a classifier;

FIG. 2 is a block diagram depicting a data processing system adapted to perform one of the aspects of the process of FIG. 1; and,

FIG. 3 is a flow chart illustrating one of the aspects of the process of FIG. 1.

### DETAILED DESCRIPTION OF THE INVENTION

Embodiments of the invention provide for synthetically generating healthcare documents for use in training a classifier. In accordance with an embodiment of the invention, a set of documents of a specific type of healthcare form are queued for processing and a specific common field is identified in each of the documents meaning that the field is present in each of the documents in the set. A statistical metric is then determined for the values in each of the documents for the common field. Optionally, statistical metric is then adjusted to a different value according to a modifier. Thereafter, the statistical metric is incorporated into a training version of the documents of the set as a value for the common field and the training version is persisted to a datastore for use as input when training a classifier adapted to classify an input document as the specific type of healthcare form.

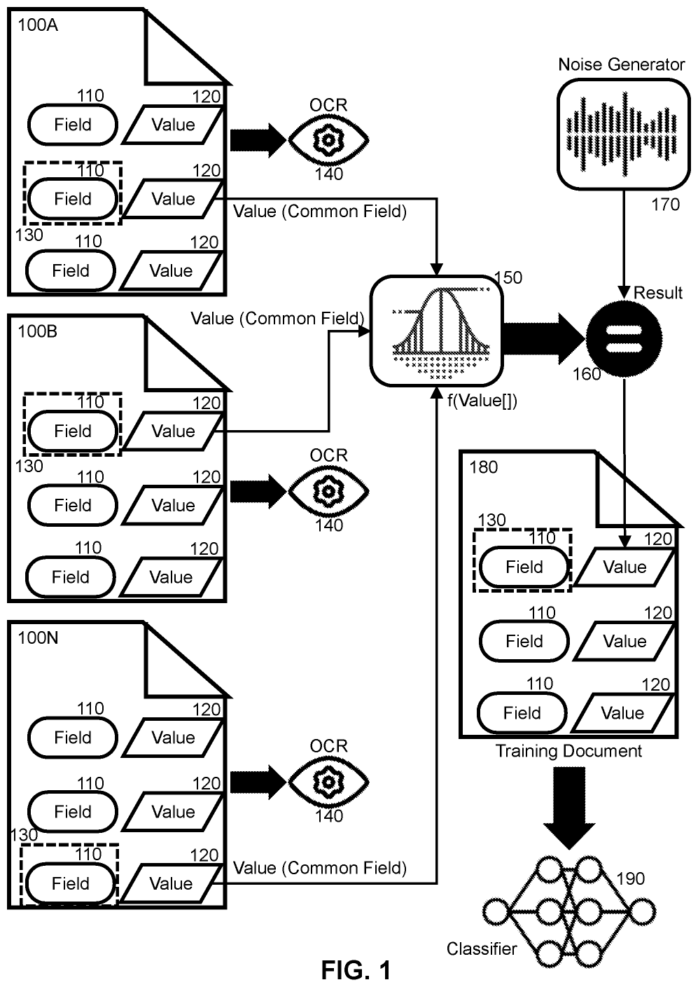

In illustration of one aspect of the embodiment, FIG. 1 pictorially shows a process of synthetically generating healthcare documents for use in training a classifier. As shown in FIG. 1, different documents 100A, 100B, 100N of similar type includes different fields 110 with corresponding values 120. The values 120 can range from numerical values to textual values. In each of the documents 100A, 100B, 100N, there are common ones 130 of the fields 110 with respective ones of the values 120. To that end, each of the documents 100A, 100B, 100N can be processed by OCR 140 in order to extract pairs of the fields 110 and respective values 120. For the common ones 130 of the fields 110, the respective values 120 are subjected to a statistical analysis 150, for instance an averaging function, a max-min function, a value accounting for a standard deviation, or other such computation.

The result 160 of the statistical analysis 150 is then modified through the introduction of random noise from noise generator 170. The modified form of the result 160 is then added to a training document 180 in connection with the common one 130 of the fields 110 and the process repeats for each other one of the common ones 130 of the fields 110. The resulting training document 180, now de-identified but contextually relevant can be used in training a document classifier 190 without risk of the divulgance of PHI.

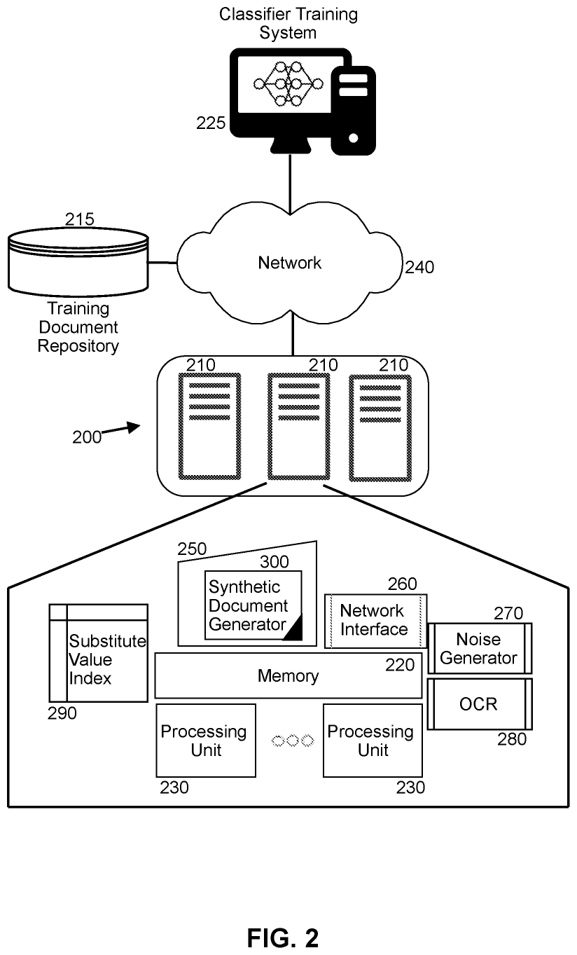

Aspects of the process described in connection with FIG. 1 can be implemented within a data processing system. In further illustration, FIG. 2 schematically shows a data processing system adapted to perform the synthetic generation of healthcare documents for use in training a classifier. In the data processing system illustrated in FIG. 1, a host computing platform 200 is provided. The host computing platform 200 includes one or more computers 210, each with memory 220 and one or more processing units 230. The computers 210 of the host computing platform (only a single computer shown for the purpose of illustrative simplicity) can be co-located within one another and in communication with one another over a local area network, or over a data communications bus, or the computers can be remotely disposed from one another and in communication with one another through network interface 260 over a data communications network 240.

An OCR processor 280 is included in the host computing platform 200 and is adapted to perform OCR on a selected document in order to store into the memory 220 a set of indexable terms present in an image of the selected document. Further, a substitute value index 290 is stored in the memory 220 and includes pairs of numeric, textual or alphanumeric values indexed according to an input numerical value so that the pairs of the values in the substitute value index 290 correlate contextually comparable terms of different values, such as different names of similar type or gender, different addresses of common region, different ages of common age grouping and the like. As an example, an input term of “Elm Street” can be converted to an index of “Seattle” which can produce as a key to the substitute value index 290, a similar term of “Maple Street” in so far as both “Elm Street” and “Maple Street” are both streets in the context of the city of Seattle. Likewise, the input term of “Mary” can be converted to an index of “Female” which can produce as a key to the substitute value index 290, a similar term of “Mable” in so far as “Mary” and “Mable” are both names in the context of the female gender.

Notably, a computing device 250 including a non-transitory computer readable storage medium can be included with the data processing system 200 and accessed by the processing units 230 of one or more of the computers 210. The computing device stores 250 thereon or retains therein a program module 300 that includes computer program instructions which when executed by one or more of the processing units 230, performs a programmatically executable process for synthetically generating healthcare documents for use in training a classifier. Specifically, the program instructions during execution invoke the OCR processor 280 upon a selected set of documents in order to generate a set of common fields in each of the documents and corresponding values for each of the common fields. The program instructions further subject the corresponding values for each of the common fields to a statistical analysis in order to produce a statistically relevant value for each one of the common fields.

The program instructions then modify each of the statistically relevant values with noise produced by noise generator 270. The program instructions then insert the modified value for each common field in an instance of the common field in a training document. Alternatively, the modified value can be used as a key to the substitute value index 290 in order to produce a substitute value for insertion into the training document in connection with the common field. In the former instance, to the extent that the statistical analysis produces an average value for the common field of an age, the average value is then modified with the noise from the noise generator 270 and inserted into an a age field in the training document.

But, in the latter instance, to the extent that the statistical analysis produces a frequency distribution of the appearance of certain words like certain street names, the most frequently appearing street name is then correlated to a particular region which is used as a key to the substitute value index 290 to locate a different street name in the same region which is then inserted into the training document as a value for the common field of street name. In any case, the program instructions then insert the training document with synthetically generated albeit contextually relevant values into a training repository 215 for use by a classifier training system 225 in training a classifier to recognize healthcare documents and the content therein.

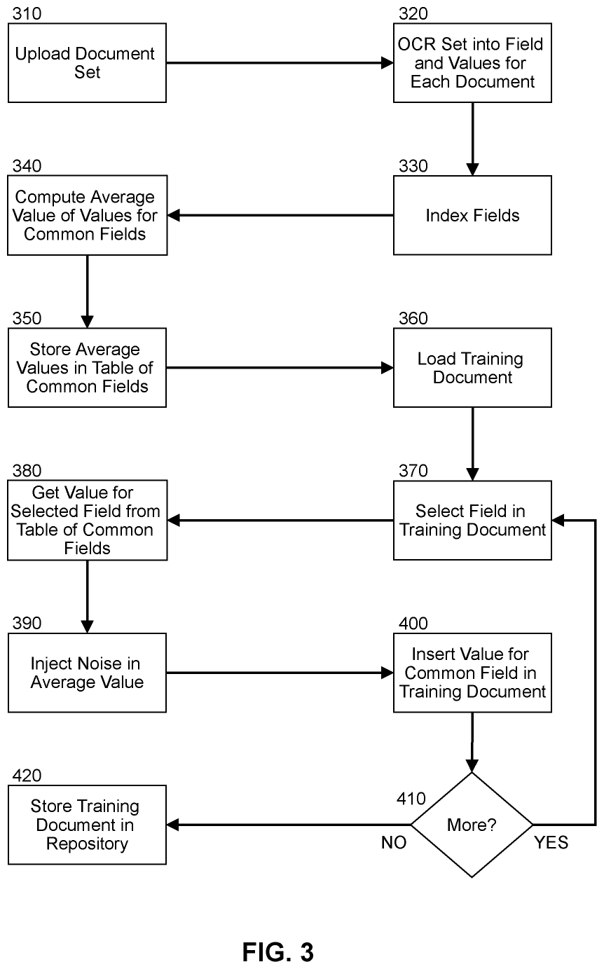

In further illustration of an exemplary operation of the module, FIG. 3 is a flow chart illustrating one of the aspects of the process of FIG. 1. Beginning in block 310, a document set of healthcare documents are uploaded for processing. Optionally, the documents conform to an annotated template including an identification of specific fields as “common fields. In block 320, each of the documents can be subjected to OCR in order to produce a set of fields and corresponding values for each of the documents. In block 330, the fields and corresponding values can be indexed and grouped together by common field type in order to identify common fields amongst the documents of the set. Then, in block 340, a statistical analysis can be performed upon the values of each common field, such as an average of numerical values, or a frequency distribution of numerical or textual values. The results of the statistical analysis for each of the common fields are then stored in a table.

In block 360, a training document is then loaded into memory for population with different values for different included fields. In block 370, a first one of the fields in the training document is selected for value population and in block 380, a corresponding value for the selected field is retrieved from the table. In block 390, random noise is injected into the retrieved value and in block 400, the resulting value is inserted into the training document in connection with the selected field. In decision block 410, if additional fields remain to be processed in connection with the training document, the next field in the training document is selected in block 370 and the process repeats through block 380. But, when no more fields remain to be processed in the training document, in block 420 the training document is uploaded to the repository for use in training a classifier of documents of similar type to the training document.

Of import, the foregoing flowchart and block diagram referred to herein illustrate the architecture, functionality, and operation of possible implementations of systems, methods, and computing devices according to various embodiments of the present invention. In this regard, each block in the flowchart or block diagrams may represent a module, segment, or portion of instructions, which includes one or more executable instructions for implementing the specified logical function or functions. In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

More specifically, the present invention may be embodied as a programmatically executable process. As well, the present invention may be embodied within a computing device upon which programmatic instructions are stored and from which the programmatic instructions are enabled to be loaded into memory of a data processing system and executed therefrom in order to perform the foregoing programmatically executable process. Even further, the present invention may be embodied within a data processing system adapted to load the programmatic instructions from a computing device and to then execute the programmatic instructions in order to perform the foregoing programmatically executable process.

To that end, the computing device is a non-transitory computer readable storage medium or media retaining therein or storing thereon computer readable program instructions. These instructions, when executed from memory by one or more processing units of a data processing system, cause the processing units to perform different programmatic processes exemplary of different aspects of the programmatically executable process. In this regard, the processing units each include an instruction execution device such as a central processing unit or “CPU” of a computer. One or more computers may be included within the data processing system. Of note, while the CPU can be a single core CPU, it will be understood that multiple CPU cores can operate within the CPU and in either instance, the instructions are directly loaded from memory into one or more of the cores of one or more of the CPUs for execution.

Aside from the direct loading of the instructions from memory for execution by one or more cores of a CPU or multiple CPUs, the computer readable program instructions described herein alternatively can be retrieved from over a computer communications network into the memory of a computer of the data processing system for execution therein. As well, only a portion of the program instructions may be retrieved into the memory from over the computer communications network, while other portions may be loaded from persistent storage of the computer. Even further, only a portion of the program instructions may execute by one or more processing cores of one or more CPUs of one of the computers of the data processing system, while other portions may cooperatively execute within a different computer of the data processing system that is either co-located with the computer or positioned remotely from the computer over the computer communications network with results of the computing by both computers shared therebetween.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A method for synthetically generating health care forms for use in training a health care form classifier, the method comprising:
receiving a multiplicity of electronic forms in memory of a host computing system;
extracting data from a specific common field located in each of the forms;
computing a statistical metric for the specific common field;
synthetically generating a value for the specific common field according to the computed statistical metric;
inserting the synthetically generated value into the specific common field of a training version of the electronic forms; and,
persisting the training version of the electronic forms as part of a training data set for a classifier adapted to classify the multiplicity of electronic forms.

2. The method of claim 1, wherein the electronic forms conform to an annotated template including an identification of the specific common field.

3. The method of claim 1, further comprising:
generating random noise; and,
modifying the synthetically generated value with the random noise.

4. The method of claim 1, wherein the computed statistical metric is a distribution of values for the specific common field.

5. A data processing system adapted for synthetically generating health care forms for use in training a health care form classifier, the system comprising:
a host computing platform comprising one or more computers, each with memory and one or processing units including one or more processing cores; and,
a synthetic form generation module comprising computer program instructions enabled while executing in the memory of at least one of the processing units of the host computing platform to perform:
receiving a multiplicity of electronic forms in memory of a host computing system;
extracting data from a specific common field located in each of the forms;
computing a statistical metric for the specific common field;
synthetically generating a value for the specific common field according to the computed statistical metric;
inserting the synthetically generated value into the specific common field of a training version of the electronic forms; and,
persisting the training version of the electronic forms as part of a training data set for a classifier adapted to classify the multiplicity of electronic forms.

6. The system of claim 5, wherein the electronic forms conform to an annotated template including an identification of the specific common field.

7. The system of claim 5, wherein the program instructions further perform:
generating random noise; and,
modifying the synthetically generated value with the random noise.

8. The system of claim 5, wherein the computed statistical metric is a distribution of values for the specific common field.

9. A computing device comprising a non-transitory computer readable storage medium having program instructions stored therein, the instructions being executable by at least one processing core of a processing unit to cause the processing unit to perform a method for synthetically generating health care forms for use in training a health care form classifier, the instructions performing:
receiving a multiplicity of electronic forms in memory of a host computing system;
extracting data from a specific common field located in each of the forms;
computing a statistical metric for the specific common field;
synthetically generating a value for the specific common field according to the computed statistical metric;
inserting the synthetically generated value into the specific common field of a training version of the electronic forms; and,
persisting the training version of the electronic forms as part of a training data set for a classifier adapted to classify the multiplicity of electronic forms.

10. The device of claim 9, wherein the electronic forms conform to an annotated template including an identification of the specific common field.

11. The device of claim 9, wherein the program instructions further perform:
generating random noise; and,
modifying the synthetically generated value with the random noise.

12. The device of claim 9, wherein the computed statistical metric is a distribution of values for the specific common field.
